(19) United States
(12) Patent Application Publication
(10) Pub. No.: US 2001/0049502 A1
(43) Pub. Date: Dec. 6, 2001

(54) GUIDE SHEATH FOR REPEATED PLACEMENT OF A DEVICE

(75) Inventor: JAMES C. CHEN, BELLEVUE, WA (US)

Correspondence Address: LAW OFFICES OF RONALD M ANDERSON, 600 108TH AVE, NE, SUITE 507, BELLEVUE, WA, 98004, US

(73) Assignee: Light Sciences Corporation

(21) Appl. No.: 09/200,292

(22) Filed: Nov. 25, 1998

(51) Int. Cl. A61M 5/00

(52) U.S. Cl. 604/167.06; 604/264

(57) ABSTRACT

A guide sheath is implanted to facilitate repeated access to a treatment site in a patient's body by a treatment device such as a probe for administering light therapy. The guide sheath reduces the risk of track contamination with bacteria, bleeding during insertion of the treatment device or a treatment evaluation probe, track metastasis, and inadvertent puncture of structures and tissues within the patient's body. The guide sheath is disposed so that its distal end is at or adjacent to the internal treatment site, and the proximal end of the guide sheath is either percutaneous or subcutaneous. The guide sheath is a thin-walled hollow tube that may be optically transparent or opaque. In one embodiment, the guide sheath has a flared opening on the proximal end to assist in inserting the treatment device into the guide sheath. This opening is covered with an self-sealing, elastomeric membrane. It is preferable to fabricate the guide sheath from a biodegradable material that will not require surgical removal. After it is no longer needed to facilitate advancing a treatment device toward the treatment site, the guide sheath can then be left in the patient's body and simply biodegrades and is absorbed. A method for inserting and a method for using the guide sheath are also disclosed.

### FIELD OF THE INVENTION

The present invention generally relates to apparatus and a method for administering medical treatment at an internal treatment site within a patient, and more specifically, to apparatus and a method employing a guide sheath or tube to enable repeated access to an internal treatment site, for delivery of light therapy to the site.

### BACKGROUND OF THE INVENTION

Using a treatment referred to as photodynamic therapy (PDT), light can be used to destroy abnormal tissue in tumors and pathogenic organisms. When administering PDT, an appropriate photoreactive agent is first infused into the patient's body or directly into abnormal tissue at a treatment site intended to be destroyed; the abnormal tissue absorbs the photoreactive agent to a much greater extent than surrounding normal tissue. Photoreactive agents used for PDT typically have a characteristic light absorption waveband and react when exposed to light within that waveband by causing the formation of singlet oxygen and the release of free radicals that destroy the abnormal tissue. Thus, when a light source producing light within the absorption waveband of the photoreactive agent is directed at the treatment site, the abnormal tissue or disease organisms at the treatment site are destroyed as a result of the effect that the light has on the photoreactive agent.

In conventional PDT, an external laser light source is used to administer light to a treatment site on the skin of a patient or at an internal treatment site that is surgically exposed. Alternatively, the light from the source may be conveyed to an internal treatment site, such as a tumor, through one or more optical fibers. Commonly assigned U.S. Pat. No. 5,445,608 discloses several different embodiments of transcutaneously implantable probes that include a plurality of relatively low intensity light sources, such as light emitting diodes (LEDs) that are usable for administering light during PDT. It has been shown that relatively low intensity light administered for an extended period of time can be even more effective in PDT than high intensity light administered for a short period of time. Thus, the light source probes disclosed in the above-referenced patent are intended to be implanted and left in place at an internal treatment site to effect PDT over an extended time.

It has also been shown that relatively low intensity light administered for an extended period of time can be so effective that tissue necrosis and associated inflammation of the target treatment area make it desirable to periodically interrupt treatment during the extended time period in order to monitor the necrosis of the target and allow any observed inflammation to subside. Following each such interruption, the PDT treatment is then resumed, as appropriate. Thus, to facilitate multiple PDT treatments and to monitor and evaluate the condition of an internal treatment site between successive treatments, it will be necessary to repeatedly access the site.

Each time that an internal treatment site is accessed poses an increased risk to the patient. Specifically, if the treatment site is accessed endoscopically, the tracks followed by the endoscopic probe may introduce contamination into the patient's body. Potentially harmful bleeding may occur during insertion of treatment devices or evaluation probes. The endoscopic probes and instruments may also cause track metastases in which cancerous cells are spread from a tumor at the treatment site along the track followed by the endoscopic devices as they are withdrawn from the patient's body. And finally, medical personnel may inadvertently puncture physiological structures, organs, vessels, or tissues while accessing the treatment site, leading to further medical complications. Accordingly, it will be apparent that it would be desirable to access internal treatment sites repeatedly and to deploy a treatment device in a manner that minimizes these risks. No currently available apparatus or procedure sufficiently minimizes the risk of such adverse effects.

Guide sheaths have been used to implant PDT devices adjacent to internal treatment sites within a patient's body. These guide sheaths are used only one time and then immediately withdrawn from the patient's body. It is also known in the medical arts to use tubes that are implanted in a patient's body for the purpose of administering fluids to, or for withdrawing fluids from, an internal site within a patient's body. These tubes can be percutaneous or subcutaneous, and typically include self-sealing elastomeric membranes. However, the prior art fails to disclose any guide sheaths that are implantable for use in enabling repeated access to an internal treatment site and to facilitate deploying a medical treatment device repeatedly at the internal treatment site. Further, all prior art guide sheaths must be surgically removed once their use is concluded. Clearly, it would be desirable to provide a guide sheath for repeatedly accessing an internal treatment site that is biodegradable and can be left in place once no further use of the guide sheath is required. While biodegradable compositions that can be left in the body are well known in the prior art, the use of such compositions for guide sheaths is not disclosed or indicated.

### SUMMARY OF THE INVENTION

The present invention relates to apparatus for repeatedly accessing an internal treatment site within a patient's body, in order to deploy a treatment device with minimal risk of concomitant medical complications. The apparatus includes a guide sheath or tube having a proximal end and a distal end. The guide sheath is preferably a hollow, thin-walled polymeric tube and may be optically transparent or opaque. The proximal end of the guide sheath may be disposed either percutaneously or subcutaneously, while the distal end of the guide sheath is disposed adjacent to or at the treatment site.

In one preferred embodiment, the guide sheath is flexible. The proximal end of the guide sheath preferably is larger than its distal end, to facilitate entry of a medical device into the interior of the guide sheath. A self-sealing elastomeric membrane is provided to cover the proximal end of the guide sheath. The walls of the guide sheath at its proximal end are fabricated of a material that is sufficiently hard to be puncture resistant. These walls preferably taper toward the hollow tube that extends to the distal end. Furthermore, the proximal end of the subcutaneously disposed guide sheath is sufficiently rigid to be palpable through a cutaneous layer of the patient's body, to facilitate relocating the guide sheath when successively accessing the treatment site.

In one embodiment, the guide sheath comprises a biodegradable polymer that does not require removal when the guide sheath is no longer required. The composition of this biodegradable polymer may be selectively varied so that the guide sheath degrades at a selected rate, e.g., over a period of time extending from several days to several months.

In another embodiment, the proximal end of the guide sheath is substantially the same size as the distal end. The proximal end of the guide sheath is cut flush with the skin and buried subcutaneously, enabling the skin to close over it.

In a variation of the embodiment that does not include a flared proximal end, the proximal end of the guide sheath is left percutaneous. In this variation, the percutaneous proximal end is preferably flexible. When it is being used to deploy a treatment device, the proximal end is flexibly aligned with the longitudinal axis of the implanted section of the guide sheath, and when not being thus used, is bent or folded over against the patient's skin, so that it is out of the way. Alternatively, this embodiment can be formed so that the proximal end substantially deviates from the longitudinal axis of the distal end, to continually conform to an outer surface of the patient's body.

In yet another embodiment, the distal end of the guide sheath is beveled or pointed so that it may be more easily passed through tissue when being inserted into a patient's body.

Another aspect of the present invention is directed to a method for implanting a guide sheath at an internal treatment site within a patient's body to facilitate repeated access to the treatment site by a treatment device. The method comprises steps that are generally consistent with the functions of the elements of the apparatus discussed above.

Finally, another aspect of the present invention is directed to a method for deploying a treatment device at an internal treatment site within a patient's body using a previously implanted guide sheath. The method comprises steps that are generally consistent with the functions of the elements of the apparatus discussed above.

Effective treatment therapy may require the use of more than one guide sheath, or the removal and repositioning of a guide sheath after multiple therapies. For instance, a large tumor being successfully treated by PDT therapy will change its size and shape as necrosis of the tumor progresses. After several treatments, it may be necessary to reposition the guide sheath to more effectively access the treatment site at the tumor.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1 is a side elevational view of an embodiment for an implanted guide sheath, in accord with the present invention, where the guide sheath is illustrated as extending into a tumor;

FIG. 2 is a side elevational view of another embodiment of a guide sheath having a proximal end disposed percutaneously, showing a PDT device in the guide sheath being used for administering light therapy to an internal treatment site within a tumor;

FIG. 3 is a side elevational view of a guide sheath having a proximal end disposed subcutaneously, showing a PDT device in the guide sheath being used for administering light therapy to the internal treatment site within a tumor;

FIG. 4 is a side elevational view of a biodegradable guide sheath having its proximal end disposed subcutaneously, and illustrating a PDT device in the guide sheath for administering light therapy to the internal treatment site in the tumor;

FIG. 5 is a side elevational view of a needle and a guide sheath, illustrating implantation of the guide sheath to facilitate repeated access to an internal treatment site by a treatment device;

FIG. 6 is a side elevational view showing a further step in the method employed for implanting the guide sheath of FIG. 5;

FIG. 7 is a side elevational view illustrating the removal of the needle shown in FIGS. 5 and 6;

FIG. 8 is a side elevational view illustrating a treatment device being advanced through the guide sheath toward an internal treatment site;

FIG. 9 is a side elevational view of an implanted subcutaneous guide sheath of the flared opening type, illustrating a needle that will be advanced to pierce the skin and a membrane covering a flared opening of the guide sheath to facilitate advancing a treatment device through the guide sheath; and

FIG. 10 is a side elevational view of an implanted subcutaneous guide sheath of the flared opening type, illustrating a needle which has been used to puncture the self-sealing membrane at the proximal end of the guide sheath to facilitate insertion of a treatment device.

### DESCRIPTION OF THE PREFERRED EMBODIMENT

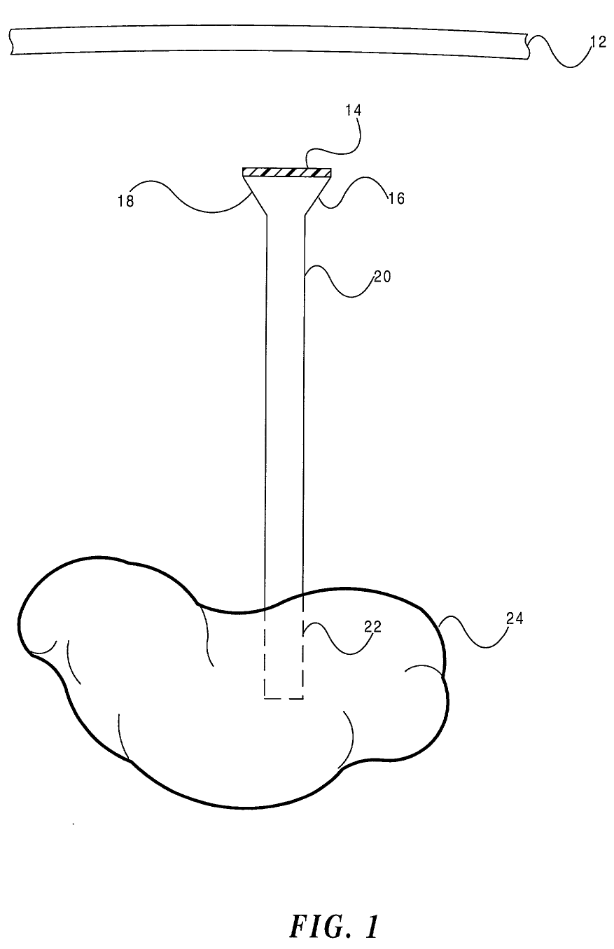

In a first embodiment of the present invention, which is shown in FIG. 1, a guide sheath 20 of a hollow, thin walled flexible polymer is placed inside a patient's body with the distal end 22 of guide sheath 20 in or adjacent to an internal treatment site; in the example illustrated, the treatment site is a tumor 24. Flexible guide sheath 20 is fabricated from a biocompatible material, such as TEFLON™, silicone, or polyurethane, so that the guide sheath can be left implanted within a patient's body for an extended period of time without any undesired effects on the body. Alternately, guide sheath 20 may be fabricated from a biocompatible and biodegradable polymer, such as polylactide compounds, so that guide sheath 20 does not have to be removed from the patient's body when it is no longer needed for guiding a treatment device to the internal treatment site. By varying the composition of the biodegradable polymer chosen for the guide sheath, the time interval required for the guide sheath to biodegrade within the patient's body can be determined. This interval can be selectively chosen, so that the guide sheath remains usable for a period ranging from days to months.

A proximal end 18 of guide sheath 20 is disposed subcutaneously below a dermal layer 12, in the example illustrated in FIG. 1. Proximal end 18 of guide sheath 20 has a flared opening 16 that is larger in diameter than distal end 22 of the guide sheath. Flared opening 16 facilitates the insertion and guiding of a needle or a treatment device (neither shown) into the proximal end of guide sheath 20. The diameter of flared opening 16 decreases to that of distal end 22 of guide sheath 20, and over most of its length, guide sheath 20 is preferably the diameter of distal end 22. The material comprising this guide sheath and any of the other embodiments discussed below can be either opaque or optically transparent or clear. If the guide sheath is to be inserted into a treatment site and used for guiding a PDT light source into place, it is preferable for the guide sheath to be clear or transparent, so that the light emitted by the PDT light source passes through the guide sheath into surrounding tissue at the treatment site. If the guide sheath is used for insertion of other types of treatment devices that do not emit light, it can be made opaque or translucent without affecting the operation of the treatment device with which it is used.

Where flared opening 16 of proximal end 18 is at its greatest diameter, it is covered with a readily puncturable, self-sealing elastomeric membrane 14. Elastomeric membrane 14 keeps bodily fluids and biological debris from passing through guide sheath 20. Because elastomeric membrane 14 is easily punctured, a needle followed by a treatment device (neither shown) can access the interior passage through guide sheath 20. In this preferred embodiment, the portion of guide sheath 20 comprising flared opening 16 is fabricated of a puncture resistant material to ensure that a sharp needle tip slides into guide sheath 20 and not through the wall of the flared opening, and is sufficiently rigid to be palpable through a cutaneous layer 12 of the patient's body, to facilitate relocating guide sheath 20 when it is necessary to successively access treatment site 24 with a treatment device. In the embodiment of FIG. 1, any repeated access of treatment site 24 by a treatment device (not shown) is accomplished very readily through guide sheath 20, with little risk of the hazards noted above in the Background of the Invention, as compared to the conventional procedure for accessing the treatment site that doesn't utilize guide sheath 20.

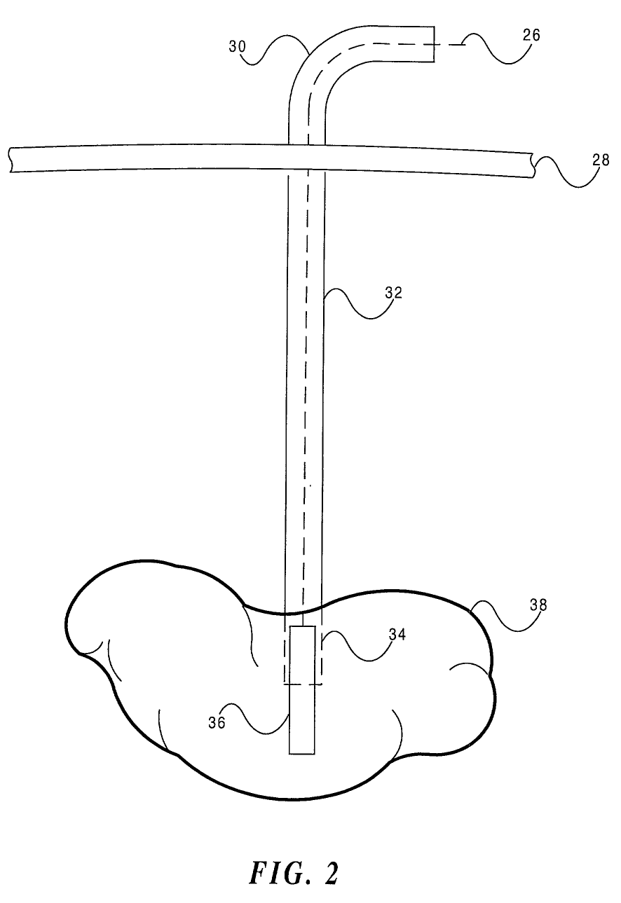

FIG. 2 illustrates an embodiment in which a proximal end 30 of a guide sheath 32 is percutaneous, extending through a dermal layer 28. This embodiment of guide sheath 32 is fabricated from a biocompatible and biodegradable polymer, although a non-biodegradable polymer could be used at least for the portion of the guide sheath that is disposed outside the patient's body, above dermal layer 28. As shown in FIG. 2, a PDT treatment device 36 (an implantable probe on which are mounted a plurality of LEDs—not specifically shown) is being used to administer light therapy interstitially to a tumor 38. PDT treatment device 36 is disposed at a distal end 34 of guide sheath 32, and power leads 26 extend from proximal end 30 of the guide sheath to an external power source (not shown).

Proximal end 30 of guide sheath 32, which is above the dermal layer 28, is preferably fabricated of a material that is sufficiently flexible to permit the proximal portion to be bent or folded over, to lie against or conform to the contour of the patient's body, i.e., against dermal layer 28 while not in use, and then be straightened to align with the longitudinal axis of the subcutaneous portion of guide sheath 32 when treatment device 36 is to be inserted through the guide sheath and advance therein toward the internal treatment site. Alternatively, proximal portion 30 of guide sheath 32, which is disposed outside the patient's body, may be formed at a generally fixed acute angle relative to the longitudinal axis of the subcutaneous portion of the guide sheath 32, so that the longitudinal axis of the proximal portion deviates substantially from that of the distal portion, proximal portion 30 being thereby adapted to lie next to an outer surface of the patient's body. The treatment device is then advanced around the bend in the guide sheath and advanced to the internal treatment site.

In the embodiment of FIG. 2, any repeated access to treatment site 38 is very readily accomplished, and when guide sheath 32 is fabricated of biodegradable material, it is unnecessary to remove the guide sheath from the patient's body after there is no further need to access the patient's body through the guide sheath. If desired, proximal portion 30 of guide sheath 32 that extends percutaneously outside the patient's body may be fabricated of a non-biodegradable material for superior durability, while the subcutaneous portion of the guide sheath 32 is fabricated from a biodegradable material having a known useful lifetime. When guide sheath 32 is no longer required, the external portion can be trimmed off, flush with the surface of dermal layer 28, and the opening through the dermal layer can be closed with sutures, surgical adhesive of staples, as appropriate. The subcutaneous portion of the guide sheath will then biodegrade and be absorbed through the normal physiological processes of the patient's body.

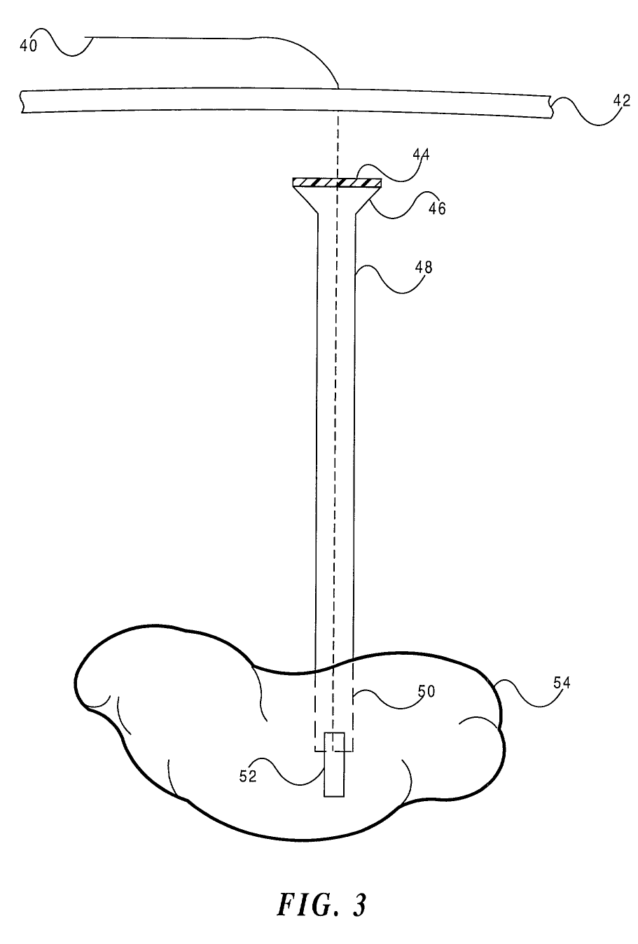

FIG. 3 illustrates a guide sheath 48 having a proximal end 46 that has a substantially larger diameter than its distal end 50. This larger diameter or flared opening at proximal end 46 is subcutaneous, but is readily located under a dermal layer 42 by palpation. The flared opening at proximal end 46 is fabricated from a biocompatible plastic or metallic material and is preferably resistant to puncturing, e.g., by the tip of a needle. However, guide sheath 48 may be fabricated completely from a biodegradable material, if desired, in which case, surgical removal of the guide sheath should not be required when access to an internal treatment site 54 through the guide sheath is no longer required. The top of the flared opening is covered with a readily puncturable, self-sealing membrane 44, fabricated, for example, from a material such as silicone. The flared opening at the proximal end facilitates the insertion of the treatment device. For the example shown in FIG. 3, a light bar 52 is shown disposed at a distal end of the guide sheath. The light bar is used for administering light to effect PDT at a treatment site within a tumor 54. Use of guide sheath 48 to repeatedly guide light bar 52 toward the treatment site in tumor 54 is accomplished with very little risk to the patient, as compared to the conventional procedure.

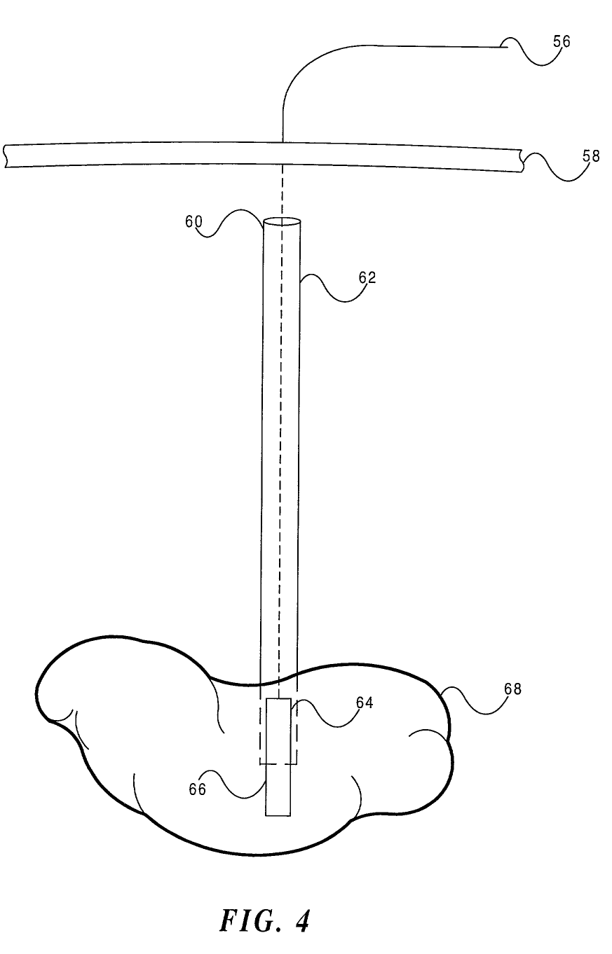

FIG. 4 illustrates an embodiment wherein a proximal end 60 of a guide sheath 62 is disposed subcutaneously below a dermal layer 58. Guide sheath 62 in this embodiment is fabricated from a biocompatible and biodegradable polymer that is optically transparent. Guide sheath 62 may be cut below dermal layer 58, or it may be implanted subcutaneously in an intact state, so that dermal layer 58 can be closed over it. A puncturable, self-sealing elastomeric membrane is preferably included at proximal end 60 of guide sheath 62 to prevent passage of bodily fluids and biological materials through the guide sheath. As depicted in FIG. 4, a PDT treatment device 66 is being used to administer light therapy at an internal treatment site within a tumor 68. An upper part 64 of PDT treatment device 66 remains inside the guide sheath; however, because the guide sheath is optically transparent, the light emitted by the upper part of the PDT treatment device is transmitted through the guide sheath and into the treatment site within tumor 68.

Note that power leads 56 extend from PDT treatment device 66 through guide sheath 62, percutaneously to an external power source (not shown). Again, in the embodiment of FIG. 4, any need for repeated access to internal treatment site 68 by PDT treatment device 66 is very readily accommodated through the guide sheath. Because guide sheath 62 is fabricated of biodegradable material, the guide sheath can be left in place after it is no longer needed to provide access to the internal treatment site in tumor 68.

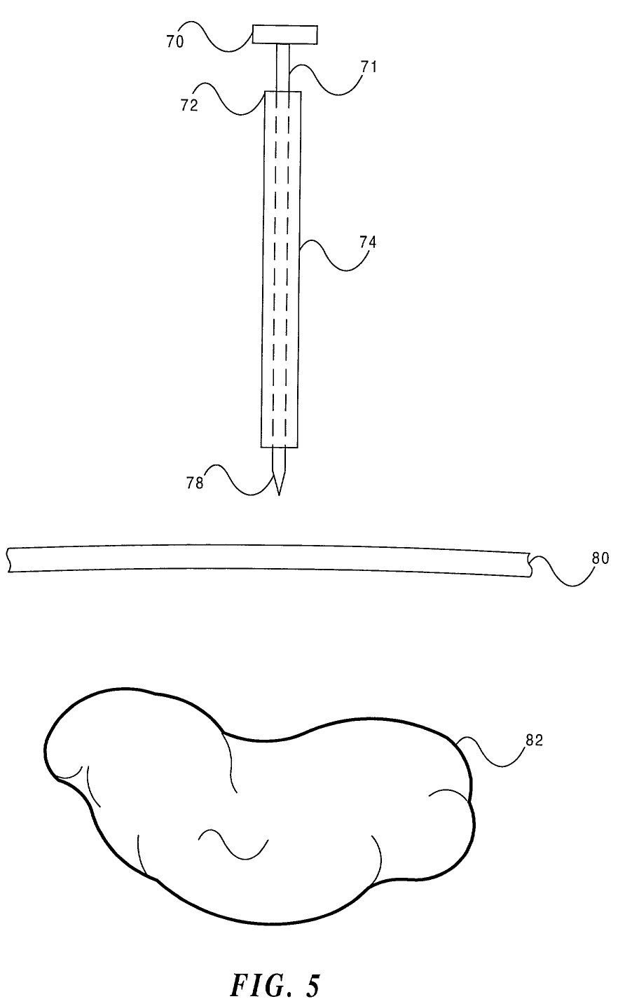

A simplified procedure for the implantation of a guide sheath that is usable to repeatedly access an internal treatment site in accord with this invention is generally illustrated in FIGS. 5-7. It is important to note that this procedure is no more invasive than the conventional procedure typically used to access an internal treatment site a single time. Once the guide sheath is in place, additional accesses of the internal treatment site by a treatment device or by a probe that is used to monitor the condition at the treatment site is accomplished with very low risk to the patient's health and well being.

FIG. 5 illustrates a first part of the procedure for implantation of a guide sheath 74. A treatment site in the patient's body has been identified, and in this example, the treatment site is at a tumor 82. A needle 71 is first inserted through guide sheath 74. A distal end 78 of the needle is beveled and is placed against a patient's skin 80, above the treatment site at tumor 82. Needle 71 is then inserted transcutaneously into the patient's body, along a path directed toward the treatment site. Guide sheath 74 is advanced over the needle, along this path. To facilitate the placement of guide sheath 74, a proximal end of needle 71 includes a flat plate 70, which is of greater diameter than that of the guide sheath. The underside of flat plate 70 contacts a proximal end 72 of guide sheath 74 to drive the guide sheath along the path followed by needle 71.

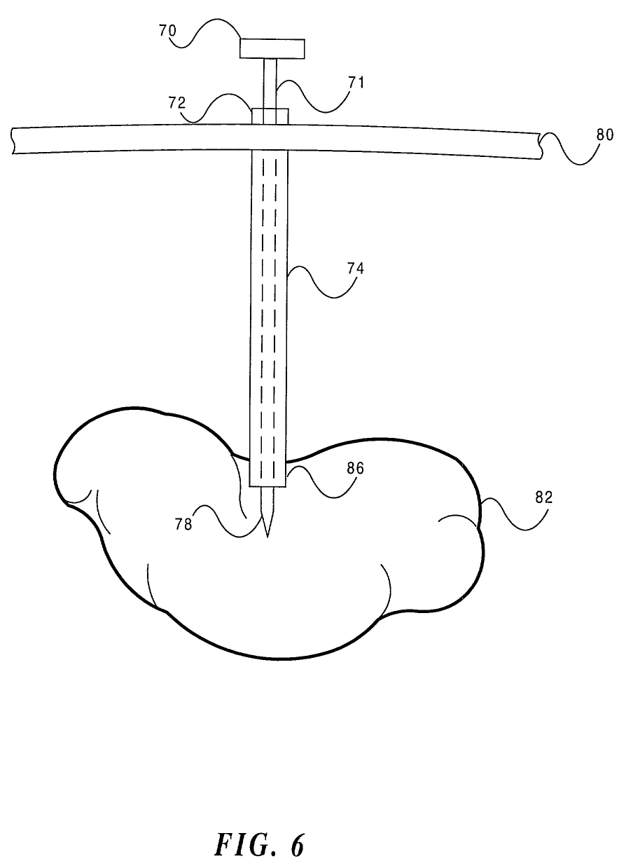

FIG. 6 illustrates the second step in this simplified procedure for the implantation of the guide sheath 74. Needle 71 is inserted into the patient's body until beveled end 78 of the needle has been advanced to the internal treatment site within tumor 82. Proximal end 72 of guide sheath 74 may be located percutaneously or subcutaneously as desired; however, FIG. 6 shows proximal end 72 of guide sheath 74 as being percutaneous (above the dermal layer 80). Note that the internal treatment site may be adjacent to rather than inside tumor 82. Real time imaging of the treatment site, e.g., using a fluoroscope or other appropriate real time imaging device, may be used to facilitate proper placement needle 71 and guide sheath 74 at the internal treatment site.

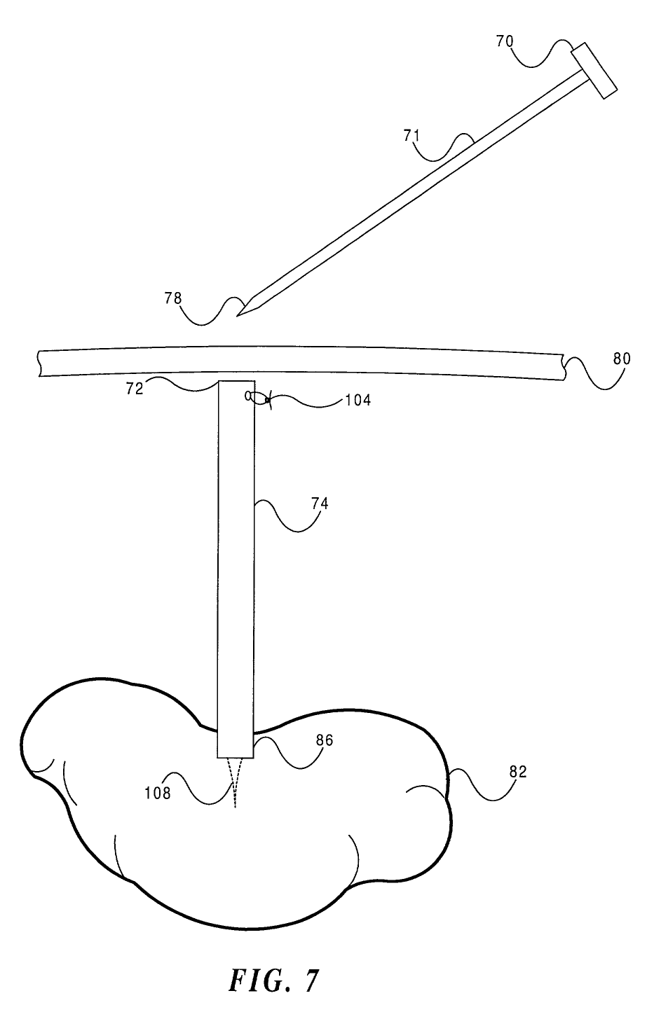

FIG. 7 illustrates the third step in this simplified procedure for the implantation of guide sheath 74. In this Figure, needle 71 has been removed, leaving guide sheath 74 implanted in the desired position, with a distal end 86 of the guide sheath disposed at the internal treatment site within tumor 82, and with proximal end 72 of the guide sheath disposed either subcutaneously, as shown, or percutaneously. A suture 104 is added at the proximal end of guide sheath 74 to hold the guide sheath in this position, preventing the guide sheath from shifting about within the patient's body due to movement by the patient. Sutures may alternatively be used to secure distal end 86 of the guide sheath to adjacent tissue or other physiological structure within the patient's body. After removal of needle 71, needle bevel 78 leaves a void or needle track 108 within tumor 82 into which a treatment device such as the PDT light bar or probe may be inserted.

Depending on the diameter of the guide sheath, the consistency of the tissue penetrated by the guide sheath, and the depth of the internal treatment site, additional steps to implant a guide sheath may be required. These steps are as follows. After inserting needle 71 percutaneously into the treatment site, it may be necessary to pass a guide wire through the needle and then remove the needle. One or more dilators of increasing diameter can then be inserted into the needle track, over the guide wire, to increase the diameter of the needle track sufficiently to accommodate the diameter of the guide sheath. The dilator(s) are then removed, and the guide sheath is passed along the dilated track until the distal end of the guide sheath is at the internal treatment site and the proximal end of the guide sheath is located percutaneously or subcutaneously as desired. The guide wire is removed. A treatment device may then be inserted and guided to the internal treatment site through the guide sheath, as discussed above. Repeated access of the treatment site can be effected using the guide sheath without incurring the risks associated with a conventional procedure that would otherwise be followed to access the treatment site.

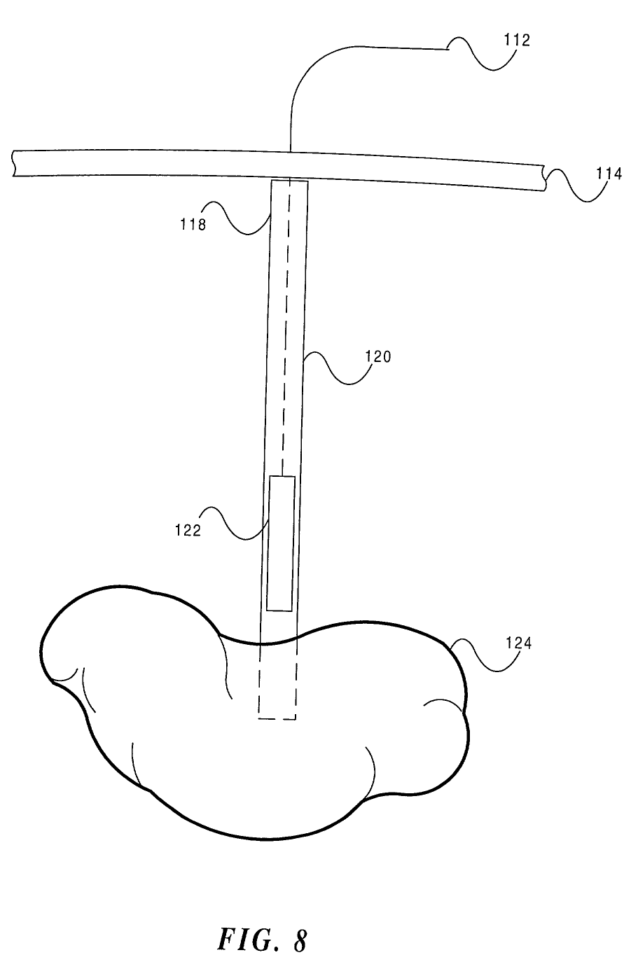

FIG. 8 illustrates the use of a guide sheath 120 inserting a PDT treatment device, i.e., a light bar 122, into an internal treatment site within a tumor 124. Power leads 112 extend from the light bar to an external power source (not shown). As depicted in FIG. 8, a proximal end 118 of guide sheath 120 is disposed subcutaneously (below a dermal layer 114). Not shown but also envisioned is a guide sheath whose proximal end is percutaneous. Since guide sheath 120 is left in place between successive accesses of the internal treatment site, light bar 122 may be removed and reinserted as required. Use of the guide sheath minimizes the risk of: (a) track contamination with bacteria introduced with the light bar, (b) excessive bleeding that might be caused by insertion of the treatment device or any evaluation probe that is inserted to determine the state of the treatment site, (c) track metastasis (i.e., the spread of abnormal cells along the track of a treatment device as the device is withdrawn from a tumor through exposed tissue), and (d) inadvertent puncture of structures and tissues within the patient's body, which may lead to further medical complications. Guide sheath 120 is preferably fabricated of biocompatible and biodegradable material, unless the medical practitioner using the guide sheath prefers to surgically remove the guide sheath after it is no longer needed for placement of a treatment device or probe at the internal treatment site.

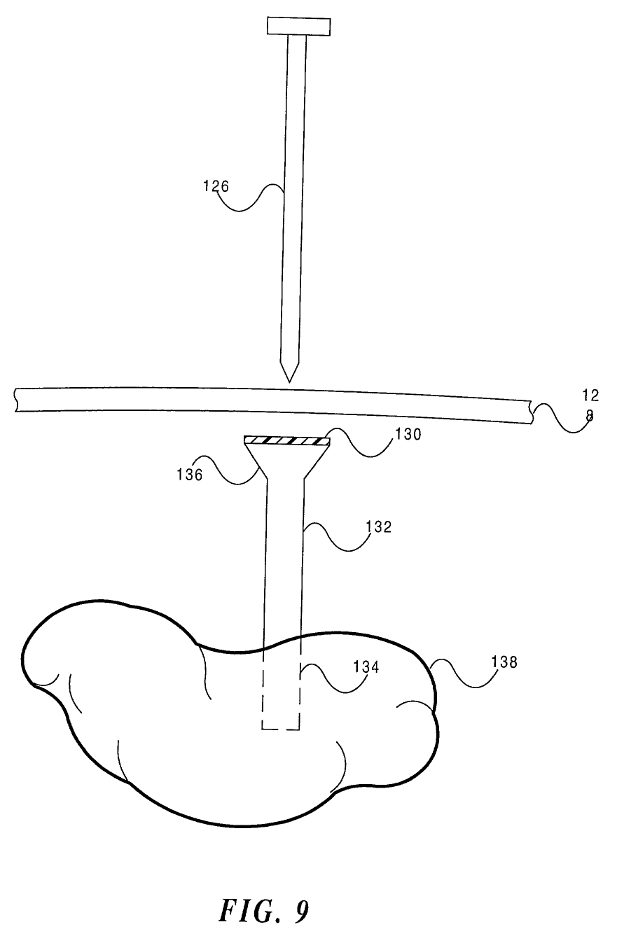

FIG. 9 illustrates an implanted, subcutaneous guide sheath 132 having a flared opening 136 and a needle 126 that is about to be inserted to provide a track into and through the flared opening to facilitate insertion of a treatment device that will be advance through the guide sheath to the internal treatment site. Flared opening 136 is palpable under a dermal layer 128, enabling the opening to be readily located so that the needle can easily be inserted, and distal end 134 of guide sheath 132 is disposed at an internal treatment site 138. Needle 126 is thus used to pierce both dermal layer 128 and a self-sealing elastomeric membrane 130 that covers flared opening 136.

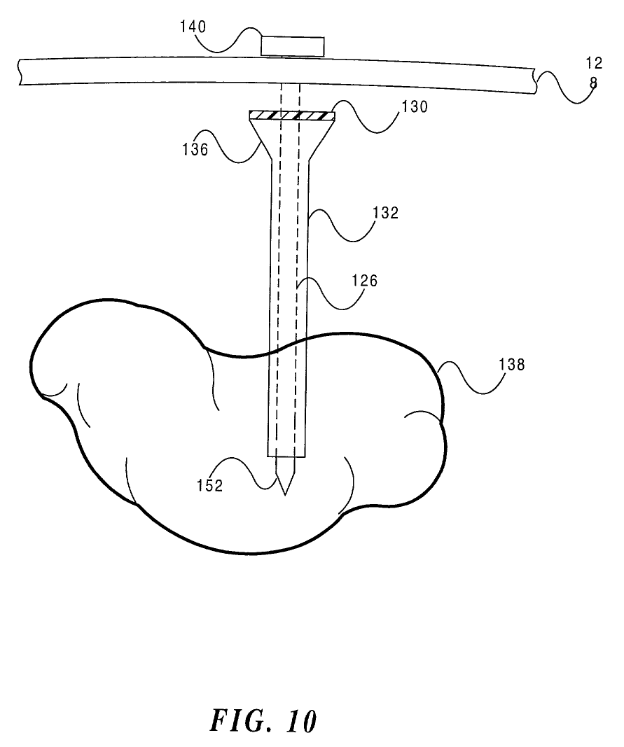

FIG. 10 shows guide sheath 132 after a beveled point 152 on needle 126 has been used to puncture self-sealing elastomeric membrane 130 and the needle advanced through the guide sheath and into tumor 138. Upon removal of the needle, a PDT or other type of treatment device may be inserted through guide sheath 132 and into internal treatment site in tumor 138. Note that the beveled point at the distal end of the needle will leave a void or needle track within the tumor into which the treatment device (not shown) may be inserted.

Although the present invention has been described in connection with the preferred form of practicing it, those of ordinary skill in the art will understand that many modifications can be made thereto within the scope of the claims that follow. Accordingly, it is not intended that the scope of the invention in any way be limited by the above description, but instead be determined entirely by reference to the claims that follow.

The invention in which an exclusive right is claimed is defined by the following:

1. Apparatus for enabling a medical device to repeatedly access an internal treatment site within a patient's body, said apparatus being adapted to be implanted and left within the patient's body for an extended period of time, comprising:
(a) a hollow guide sheath having a distal end and a proximal end, said distal end being adapted to be disposed adjacent to said internal treatment site; and
(b) a self-sealing, puncturable, elastomeric membrane disposed adjacent to the proximal end of the guide sheath, said elastomeric membrane closing the guide sheath to prevent bodily fluids from freely flowing through the guide sheath while enabling a medical device to be inserted through said elastomeric membrane by puncturing said elastomeric membrane, said guide sheath guiding the medical device to and from the internal treatment site.

2. The apparatus of claim 1, wherein the guide sheath comprises a substantially optically transparent material.

3. The apparatus of claim 1, wherein the guide sheath comprises a flexible, biocompatible polymer.

4. The apparatus of claim 1, wherein the guide sheath comprises a flexible, biodegradable material that is adapted to remain within the patient's body after the guide sheath is no longer needed for accessing the internal treatment site, and to then biodegrade so that it is unnecessary to remove the guide sheath from the patient's body.

5. The apparatus of claim 1, wherein the guide sheath is larger at its proximal end than at its distal end, to facilitate entry of a medical device into an interior of the guide sheath.

6. The apparatus of claim 5, wherein at least a portion of the guide sheath disposed adjacent to the proximal end of the guide sheath comprises a puncture resistant material.

7. The apparatus of claim 1, wherein said elastomeric membrane covers an opening into the proximal end of the guide sheath.

8. The apparatus of claim 1, wherein the guide sheath is adapted to be disposed subcutaneously within the patient's body.

9. The apparatus of claim 8, wherein the proximal end of the guide sheath comprises a material that is sufficiently rigid to be palpable through a cutaneous layer of the patient's body, to facilitate relocating the guide sheath when successively accessing the internal treatment site through the guide sheath.

10. The apparatus of claim 1, wherein the distal end of the guide sheath is beveled to facilitate insertion of the guide sheath through tissue of the patient's body.

11. The apparatus of claim 1, wherein the treatment device comprises a probe for administering light therapy to the internal treatment site.

12. The apparatus of claim 1, wherein the guide sheath is adapted to extend percutaneously from the internal treatment site, so that said proximal end of the guide sheath is disposed outside the patient's body.

13. The apparatus of claim 12, wherein the guide sheath has a longitudinal axis along which the distal end of the guide sheath extends, said proximal end being sufficiently flexible to deviate substantially from the longitudinal axis and thus adapted to conform to an outer surface of the patient's body.

14. Apparatus for providing access to an internal treatment site within a patient's body, said apparatus being designed to be implanted and left within a patient for an extended period of time, for the purpose of facilitating repeated access to the internal treatment site by a treatment device, said apparatus comprising: a hollow guide sheath having a distal end and a proximal end, said distal end being adapted to be disposed at or adjacent to said internal treatment site, said guide sheath being fabricated at least in part of a biodegradable material that is adapted to remain disposed within the patient's body and to biodegrade after the guide sheath is no longer used for providing access to the internal treatment site by the treatment device, so that it is unnecessary to remove at least said part of the guide sheath from the patient's body.

15. The apparatus of claim 14, wherein the guide sheath comprises a substantially optically transparent material.

16. The apparatus of claim 14, wherein at least said part of the guide sheath is biocompatible.

17. The apparatus of claim 14, wherein the guide sheath comprises a substantially flexible material.

18. The apparatus of claim 14, wherein the guide sheath is adapted to extend percutaneously from the internal treatment site so that said proximal end of the guide sheath is disposed outside the patient's body, at least said portion of the guide sheath that is biodegradable being disposed within the patient's body.

19. The apparatus of claim 18, wherein said proximal end of the guide sheath is adapted to remain outside the patient's body and is not biodegradable.

20. The apparatus of claim 18, wherein the guide sheath has a longitudinal axis along which the distal end of the guide sheath extends, said proximal end being sufficiently flexible to deviate substantially from the longitudinal axis and thus adapted to conform to an outer surface of the patient's body.

21. The apparatus of claim 14, wherein the distal end of the guide sheath is beveled to enable insertion of the guide sheath through tissue in the patient's body to position the distal end at or adjacent to the internal treatment site.

22. The apparatus of claim 14, wherein the treatment device comprises a probe for administering light therapy to the internal treatment site.

23. The apparatus of claim 14, wherein the guide sheath is larger at its proximal end than at its distal end, to facilitate insertion of the treatment device into an interior of the guide sheath.

24. The apparatus of claim 23, wherein at least a portion of the guide sheath disposed adjacent to the proximal end of the guide sheath comprises a puncture resistant material.

25. The apparatus of claim 14, wherein the guide sheath includes a self-sealing, puncturable elastomeric membrane that closes the guide sheath to prevent bodily fluids from freely flowing through the guide sheath, while enabling the treatment device to be inserted through the guide sheath, by puncturing said elastomeric membrane.

26. The apparatus of claim 25, wherein said elastomeric membrane is disposed adjacent to the proximal end of the guide sheath.

27. The apparatus of claim 14, wherein the guide sheath is adapted to be disposed subcutaneously within the patient's body.

28. The apparatus of claim 27, wherein the proximal end of the guide sheath comprises a material that is sufficiently rigid to be palpable through a cutaneous layer of the patient's body, to facilitate relocating the guide sheath when successively accessing the treatment site.

29. The apparatus of claim 27, wherein the guide sheath includes a self-sealing, puncturable elastomeric membrane that closes the guide sheath to prevent bodily fluids from freely flowing through the guide sheath, while enabling the treatment device to be inserted through said guide sheath by puncturing said elastomeric membrane.

30. The apparatus of claim 29, wherein said elastomeric membrane covers the proximal end of the guide sheath.

31. A method for implanting a guide sheath used for accessing an internal treatment site within a patient's body, to facilitate repeated access to the internal treatment site by a treatment device, comprising the steps of:
(a) providing a needle;
(b) providing a guide sheath having a proximal end and a distal end;
(c) inserting the needle through the guide sheath;
(d) inserting the needle transcutaneously into the patient's body and advancing the needle toward the internal treatment site through tissue in the patient's body;
(e) forcing the distal end of the guide sheath into the patient's body over the needle and advancing the guide sheath toward the internal treatment site, the proximal end of the guide sheath being disposed percutaneously, and the distal end of the guide sheath being disposed adjacent to the internal treatment site;
(f) removing the needle, leaving the guide sheath in place for guiding the treatment device to the internal treatment site; and
(g) affixing the guide sheath in place, to prevent its movement relative to the treatment site and to facilitate repetitively guiding the treatment device to the internal treatment site over an extended period of time.

32. The method of claim 31, wherein at least a portion of the guide sheath comprises a biodegradable material that is adapted to remain disposed within the patient's body after the guide sheath is no longer used for guiding the treatment device to the internal treatment site, and to biodegrade within the patient's body so that it is unnecessary to remove at least said portion of the guide sheath from the patient's body.

33. The method of claim 31, wherein the guide sheath includes a self-sealing, puncturable elastomeric membrane that closes the guide sheath to prevent bodily fluids from freely flowing through the guide sheath, while enabling a medical device to be inserted through said guide sheath by puncturing said elastomeric membrane.

34. The method of claim 31, further comprising the steps of:
(a) providing a guide wire;
(b) providing a dilator;
(c) inserting the guide wire through said needle after the needle has been advance toward the treatment site;
(d) removing the needle from the tissue in the patient's body and inserting the dilator over the guide wire;
(f) expanding the dilator to increase a passage through the tissue;
(g) removing the dilator;
(h) forcing the distal end of the guide sheath into the patient's body over the guide wire and advancing the guide sheath toward the internal treatment site, the proximal end of the guide sheath being disposed percutaneously, and the distal end of the guide sheath being disposed adjacent to the internal treatment site; and
(i) removing the guide wire, leaving the guide sheath in place.

35. The method of claim 31, further comprising the steps of:
(a) cutting the proximal end of the guide sheath flush with the skin of the patient's body; and
(b) closing the skin over the proximal end of the guide sheath that was cut.

36. The method of claim 31, further comprising the step of positioning the proximal end of the guide sheath subcutaneously such that when it is next necessary to use the guide sheath to advance the treatment device to the internal treatment site, the proximal end is palpable under the skin of the patient.

37. The method of claim 36, wherein the guide sheath comprises a biodegradable material that is adapted to remain disposed within the patient's body after the guide sheath is no longer used for guiding the treatment device to the internal treatment site, and to biodegrade within the patient's body so that it is then unnecessary to remove the guide sheath from the patient's body.

38. The method of claim 31, further comprising the step of imaging the treatment site within the patient's body to facilitate the placement of the guide sheath.

39. The method of claim 31, wherein the step of affixing comprises the step of attaching the proximal end of the guide sheath to the patient's body with absorbable sutures to prevent migration of the guide sheath.

40. The method of claim 31, wherein the step of affixing comprises the step of attaching the distal end of the guide sheath to the patient's body with absorbable sutures to prevent migration of the guide sheath.

41. A method for deploying a medical device at an internal treatment site within a patient's body, using a previously implanted guide sheath, comprising the steps of:
(a) providing an identified treatment site, a guide sheath that is disposed subcutaneously within the patient's body, a needle, and a treatment device;
(b) locating a proximal end of the guide sheath within the patient's body;
(c) inserting said needle transcutaneously into the proximal end of the guide sheath and advancing the treatment device over the needle toward the internal treatment site through the guide sheath;
(d) removing said needle; and
(e) removing said treatment device from the guide sheath and the patient's body after the treatment device has performed a desired function.

42. The method of claim 41, further comprising the step of imaging the treatment site within the patient's body to facilitate the placement of the treatment device at the internal treatment site.

43. The method of claim 41, wherein the treatment device is employed to administer light therapy to the internal treatment site.
